(19) United States
(12) Patent Application Publication
(10) Pub. No.: US 2026/0314990 A1
(43) Pub. Date: Oct. 15, 2026

(54) TRANSIENT-PHASE EMG SIGNAL GESTURE RECOGNITION METHOD BASED ON EMG ACTIVATION INTENSITY TRANSITION FEATURE AND ADAPTIVE ANALYSIS WINDOW LENGTH

(71) Applicant: SOUTHEAST UNIVERSITY, Jiangsu (CN)

(72) Inventors: Baoguo XU, Jiangsu (CN); Shipeng REN, Jiangsu (CN); Wenqian CAO, Jiangsu (CN); Aiguo SONG, Jiangsu (CN)

(73) Assignee: SOUTHEAST UNIVERSITY, Jiangsu (CN)

(21) Appl. No.: 19/554,647

(22) Filed: Mar. 3, 2026

Related U.S. Application Data
(63) Continuation of application No. PCT/CN2025/105440, filed on Jun. 30, 2025.

(30) Foreign Application Priority Data
Apr. 15, 2025 (CN) 202510467379.5

(51) Int. Cl. A61B 5/397; A61B 5/00; G06F 3/01; G06N 20/10

(52) U.S. Cl. CPC A61B 5/397; A61B 5/7267; G06F 3/015; G06F 3/017; G06N 20/10

(57) ABSTRACT

Provided is a transient-phase EMG signal gesture recognition method based on an EMG activation intensity transition feature and an adaptive analysis window length. The method performs signal acquisition and preprocessing, onset time detection, signal frame formatting and feature extraction, and trains SVM classifiers in an offline mode. In an online mode, the method acquires preprocessed signals, performs onset time detection and signal frame formatting, extracts the EMG activation intensity transition feature and inputs the same to the offline-trained SVM classifier, such that the analysis window length is dynamically adjusted based on the confidence level, and a confidence threshold is adjusted in real time.

### CROSS-REFERENCE TO RELATED APPLICATION

This application is a continuation application of international application of PCT application serial no. PCT/CN2025/105440, filed on Jun. 30, 2025, which claims the priority benefit of China application no. 202510467379.5, filed on Apr. 15, 2025. The entirety of each of the above mentioned patent applications is hereby incorporated by reference herein and made a part of this specification.

### TECHNICAL FIELD

The present disclosure relates to the field of biological signal processing and prediction technology, in particular to the technology of using a transient phase of electromyography signals for motion intention prediction, which is widely applicable to the fields, such as prosthetic control and motor rehabilitation devices.

### BACKGROUND

Electromyography (EMG) signals are bioelectrical signals generated on the skin surface during muscle contraction and contain rich information about muscle activity, and these signals can be used for predicting motion intention. Conventional EMG signal processing methods typically rely on signals in a steady state for feature extraction. However, transient-phase signals (that is, signals corresponding to the transition process from rest to movement) contain important motion intention information and are particularly critical for improving the speed of motion intention recognition. Nevertheless, existing techniques that utilize transient-phase signals are generally based on fixed-length analysis windows, which have the following problems:

Imbalance between spatial feature representation and computational cost: Existing analysis methods either rely on conventional time-domain features that analyze discrete channel signals with no spatial features or employ deep learning models such as convolutional neural networks (CNNs) and long short-term memory (LSTM) networks, which have relatively high computational costs. Therefore, it is difficult to obtain spatial feature information while maintaining low computational costs.

Imbalance between recognition accuracy and response speed: When the length of the analysis window is extended, recognition accuracy can be improved, but the response speed is reduced. Conversely, when the length of the analysis window is shortened, the response speed is improved at the expense of recognition accuracy. Therefore, a fixed-length analysis window cannot simultaneously achieve both high recognition accuracy and fast response speed.

Limited algorithm generalization across individuals and tasks: Existing EMG signal processing methods are typically based on fixed analysis window lengths, which cannot adapt to changes across different individuals and tasks.

The present disclosure provides a gesture recognition method capable of dynamically adjusting an analysis window length, and adaptively adjusting the analysis window length for different individuals and different tasks, thereby achieving a balance between recognition accuracy and response speed through confidence evaluation.

### SUMMARY

The present disclosure provides a transient-phase EMG signal gesture recognition method based on an EMG activation intensity transition feature and an adaptive analysis window length.

The method can adaptively adjust the analysis window length for different individuals and different tasks, achieving a balance between recognition accuracy and response speed by evaluating a confidence level.

In order to achieve the above objectives, the present disclosure implements the following technical solutions: a transient-phase EMG signal gesture recognition method based on an EMG activation intensity transition feature and an adaptive analysis window length, including the following steps:

S1: Acquiring and preprocessing signals in an offline mode: EMG signals are acquired and preprocessed offline using a high-density EMG sensor.

S2: Performing onset time detection in the offline mode: Obtaining onset time of motion intention T0_{offline} and an onset threshold mEMGₒₙₛₑₜ for subsequent online detection algorithms using a detEMG-based offline signal onset TO detection algorithm.

S3: Performing signal frame formatting in the offline mode: Frame formatting is performed on the signals throughout a transient phase following the onset time TO.

S4: Extracting a feature in the offline mode: The EMG activation intensity transition (directional activation intensity transition histogram) features for each signal frame are sequentially calculated.

S5: Training SVM classifiers: a plurality of classifiers corresponding to different analysis window lengths are trained in this stage.

S6: Acquiring and preprocessing signals in an online mode: EMG signals are acquired and preprocessed online using the high-density EMG sensor.

S7: Performing signal onset time detection in the online mode: Onset time of motion intention is identified by adopting an mEMGₒₙₛₑₜ-based onset time T0ₒₙₗᵢₙₑ detection algorithm for an online signal.

S8: Performing signal frame formatting in the online mode: Frame formatting is performed on the signals throughout a transient phase following the onset time T0ₒₙₗᵢₙₑ.

S9: Extracting a feature in the online mode: An EMG activation intensity transition (directional activation intensity transition histogram) feature is calculated for each signal frame.

S10: Inputting to an offline SVM classifier: Prediction type and confidence level of a current frame are calculated, and the analysis window length is adjusted dynamically.

S11: Performing real-time feedback and adjustment: A confidence threshold is adjusted according to the real-time results.

Further, the high-density EMG sensor in S1 needs to provide EMG signals from 64 or more channels, so as to capture electrical activity from different muscle regions.

Further, a method for preprocessing the signals in S1 includes the following steps:

S1-1: Performing 50 Hz notch filtering: power-line interference is removed.

S1-2: Performing band-pass filtering (30 Hz-500 Hz): Muscle characteristics are simulated to remove low-frequency and high-frequency noises, and retain valid EMG signals.

Further, the onset time detection in the offline mode is performed using the mEMGₒₙₛₑₜ-based onset time TO detection algorithm in S2. detEMG of the signal is calculated using the algorithm to find onset time of sudden changes in the signal, thereby identifying the onset of motion intention. Specific steps are as follows:

S2-1: First, summing data from all channels, which is equivalent to compressing the high-density EMG signals into a single channel. On this basis, detEMG is calculated according to the following formula:

$${mEMG}_{i} = \frac{1}{N} {\sum }_{j = i}^{N + i - 1} \vert {x}_{j} \vert$$

$${detEMG}_{i} = {mEMG}_{i} - \frac{1}{{T}_{det}} {\int }_{{t}_{i} - {T}_{det}}^{{t}_{i}} {mEMG}_{i}$$

where N denotes a total number of data points; |xⱼ| denotes an absolute value of an i^{th} data point; mEMGᵢ denotes an EMG activation intensity value at the i^{th} data point; T_{det} denotes detection span time; and detEMGᵢ denotes a detection signal at the i^{th} data point.

S2-2: Calculating a time point at which the detEMGᵢ reaches 0.5 times its maximum value, and defining the same as the onset time of offline motion intention T0_{offline}.

S2-3: Obtaining mEMGᵢ at the time point simultaneously, and recording as mEMGₒₙₛₑₜ, where the value will be subsequently used for online recognition.

Further, a method for signal frame formatting throughout the transient phase in S3 is as follows: starting from T0, signals of each channel are averaged over a fixed time interval ΔT between a t(i−1)^{th} time moment and a t(i) time moment, and the results are defined as an i^{th} frame signal (i=1,2,3 . . . f_sum). The specific calculation formula is as follows:

$${F}_{i} = \frac{1}{Δ t} {\int }_{{t}_{i} - Δ t}^{{t}_{i}} {mEmg}_{i}$$

where Fᵢ denotes the i^{th} frame signal (multi-channel data; it has an 8×8 matrix format when a sensor size is 8×8), and tᵢ=T₀+i×Δt denotes ending time of an i^{th} frame.

f_sum denotes a total number of frames throughout the transient phase, with a calculation formula as follows:

$$f\_sum = \frac{{T}_{transient}}{Δ t}$$

where Δt denotes a time interval, and Tₜᵣₐₙₛᵢₑₙₜ denotes a duration of the entire transient phase, which remains consistent with the offline mode.

Further, a method for extracting a feature in the offline mode in S4 is as follows: EMG activation intensity transition (directional activation intensity transition histogram) features for each signal frame are sequentially calculated. Textural information from signals within local regions is extracted from the EMG activation intensity transition feature, making it suitable for describing spatial features of EMG signals. Moreover, the EMG activation intensity transition feature has a strong capability to capture changes in muscle activity patterns. The specific calculation steps are as follows:

S4-1: Calculating the EMG activation intensity transition feature.

First, for each EMG sensor channel in the current frame, EMG activation intensity transition values in a horizontal direction STₓ and a vertical direction ST_{y} are calculated:

STₓ=mEMG(x+1,y)−mEMG(x−1,y)

ST_{y}=mEMG(x,y+1)−mEMG(x,y−1)

where mEMG(x, y) denotes an EMG activation intensity value at an EMG sensor channel (x, y) in the current frame;

an activation intensity transition magnitude ST and an activation intensity transition direction θ for each EMG sensor channel (x, y) are then calculated:

$$S T = \sqrt{S {T}_{x}^{2} + S {T}_{y}^{2}}$$

$$θ = {tan}^{- 1} \frac{S {T}_{y}}{S {T}_{x}}$$

S4-2: Constructing an EMG activation intensity transition histogram

The current frame is divided into a plurality of sub-frames, and each sub-frame contains 2×2 channels. Within each sub-frame, an activation intensity transition histogram is calculated based on the activation intensity transition direction of each channel. Specific steps are as follows:

S4-2-1: Dividing the activation intensity transition direction θ (0-180°) into a plurality of intervals.

S4-2-2: Assigning the activation intensity transition magnitude ST of each EMG sensor channel to a corresponding histogram bin according to its activation intensity transition direction θ. Specifically, when the activation intensity transition direction θ of the EMG sensor channel falls within a k^{th} interval, the activation intensity transition magnitude ST of the EMG sensor channel is added to a histogram of the k^{th} interval.

S4-2-3: Converting the histogram into a feature vector, and obtaining a final EMG activation intensity transition feature vector.

Further, steps for training SVM classifiers in S5 are as follows:

S5-1: Classifying as training sets and test sets

All gesture sample data are classified as training sets and test sets according to a predetermined ratio, which may be selected within a range from 1:4 to 1:2.

S5-2: Inputting signals

Starting from an m^{th} frame (initial input frame), EMG activation intensity transition features from the m^{th} frame to an (m+1)^{th} frame are averaged across dimensions and input into the SVM classifiers.

S5-3: Obtaining prediction results and calculating confidence: for each gesture category, the SVM classifier outputs a prediction result and a corresponding confidence level, where the confidence level represents the reliability of the prediction result.

S5-4: Adjusting signal input: an analysis window length range of the SVM classifier is continuously extended, and S5-3 and S5-4 are repeated until all frames within the entire transient phase are covered.

Further, a method for preprocessing the signals in S6 includes the following steps:

S6-1: Performing 50 Hz notch filtering: power-line interference is removed.

S6-2: Performing band-pass filtering (30 Hz-500 Hz): Muscle characteristics are simulated to remove low-frequency and high-frequency noises, and retain valid EMG signals.

Further, steps for adopting an EMGₒₙₛₑₜ-based onset time T0ₒₙₗᵢₙₑ detection algorithm in S7 are as follows:

S7-1: First, summing data from all channels, which is equivalent to compressing the high-density EMG signals into a single channel. On this basis, mEMGᵢ is calculated according to the following formula:

$${mEMG}_{i} = \frac{1}{N} {\sum }_{j = i}^{N + i - 1} \vert {x}_{j} \vert$$

where N denotes a total number of data points; |xⱼ| denotes an absolute value of an i^{th} data point; and mEMG; denotes an EMG activation intensity value at the i^{th} data point.

S7-2: Comparing a current mEMGᵢ and mEMGₒₙₛₑₜ (a threshold at the onset time mEMGᵢ) until the current mEMGᵢ is greater than or equal to mEMGₒₙₛₑₜ, and marking the corresponding time point as an onset time T0ₒₙₗᵢₙₑ.

Further, a method for signal frame formatting in S8 is specifically as follows: starting from T0ₒₙₗᵢₙₑ, signals of each channel are averaged over a fixed time interval ΔT between a t(i−1)^{th} time moment and a t(i) time moment, and the results are defined as an i^{th} frame signal (i=1, 2, 3 . . . f_sum), where f_sum denotes a total number of frames throughout the transient phase. Refer to S3 for the specific calculation formula.

Further, a method for extracting a feature in the online mode in S9 is as follows: An EMG activation intensity transition (directional activation intensity transition histogram) feature is calculated for each signal frame. Refer to S4 for the specific calculation method and formula.

Further, specific steps for inputting to an offline SVM classifier in S10 are as follows:

S10-1: Calculating prediction type and confidence level of a current frame.

Starting from an m^{th} frame (initial input frame), EMG activation intensity transition features from the m^{th} frame to an m+1^{th} frame are averaged across dimensions and input into the corresponding offline SVM classifier. A current prediction Result and its confidence level Confidence are then calculated.

S10-2: Adjusting the analysis window length dynamically. The confidence level Confidence obtained in the previous step is compared with a predefined confidence threshold Confidence_{accept}. When the confidence level reaches the predefined acceptable confidence level, the current prediction result is output, and further data acquisition is terminated. When the confidence level does not reach the predefined acceptable confidence level, a length of the input signal analysis window is increased, and S10-1 is repeated until the confidence level reaches the acceptable confidence level.

S10-3: Outputting results: the recognition result is output, and further data acquisition is terminated. When the input signal length has already covered the entire transient phase, the recognition result is also output.

Further, a specific method for performing real-time feedback and adjustment in S11 is as follows:

S11-1: Presetting an initial confidence threshold Confidence_{accept_0}

S11-2: Adjusting a confidence threshold Confidence_{accept_new} according to the real-time results to meet the needs of different individuals and tasks, thereby dynamically adjusting signal acquisition and analysis strategies and achieving a balance between recognition accuracy and response speed.

Compared with the prior art, the present disclosure has the following advantages:

1. Reducing the computational costs: Existing analysis methods that preserve spatial information typically rely on expensive deep learning models, such as convolutional neural networks (CNNs) and long short-term memory (LSTM) networks. In contrast, the present disclosure employs the EMG activation intensity transition feature, which can significantly reduce the computational costs.

2. Improving response speed: Compared with other EMG-based gesture recognition solutions, the present disclosure directly processes transient-phase EMG signals before signal stabilization, such that gesture recognition can be performed approximately 400 ms earlier.

3. Balance between recognition accuracy and response speed: The present disclosure provides a gesture recognition method capable of dynamically adjusting an analysis window length. The method can adaptively adjust the analysis window length for different individuals and different tasks, achieving a balance between recognition accuracy and response speed by evaluating a confidence level.

### BRIEF DESCRIPTION OF THE DRAWINGS

FIG. 1 is a schematic flowchart of a method according to the present disclosure.

FIG. 2 is a schematic structural diagram of onset time detection results according to an embodiment of the present disclosure.

FIG. 3 is a schematic diagram illustrating changes in analysis window length and average recognition accuracy with different confidence threshold settings according to an embodiment of the present disclosure.

### DETAILED DESCRIPTIONS OF THE EMBODIMENTS

The implementation modes of the present disclosure will be further described in detail with reference to the accompanying drawings and the embodiments. The following embodiments are used for describing the present disclosure, but are not used for limiting the scope of the present disclosure.

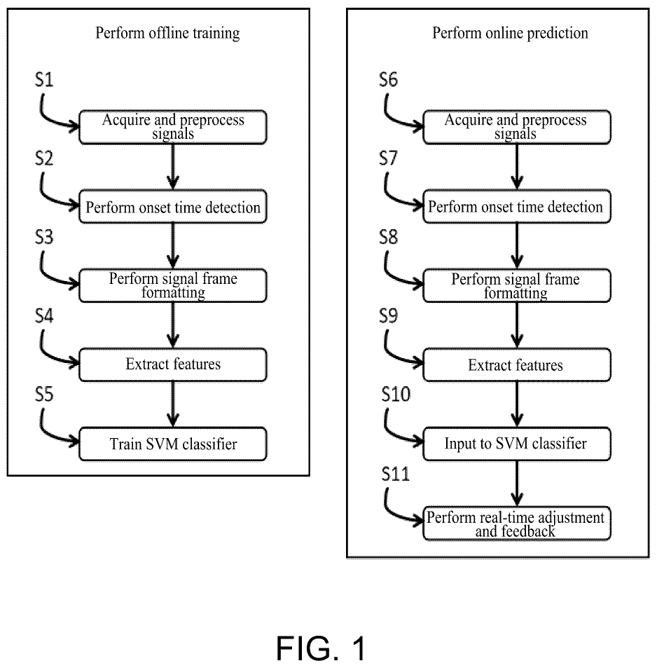

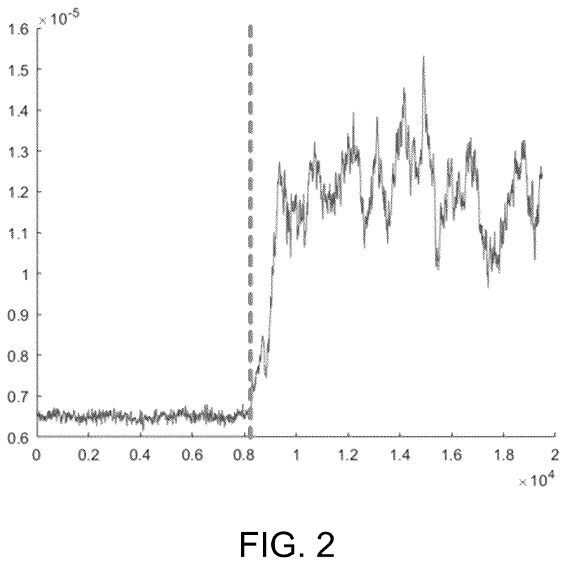

Embodiment: Referring to FIGS. 1-3, a transient-phase EMG signal gesture recognition method based on an EMG activation intensity transition feature and an adaptive analysis window length is provided. The method includes the following steps.

S1: Acquiring and preprocessing signals in an offline mode. Gesture classification data are acquired using an open-source dataset, and the dataset is acquired by using a 128-channel high-density EMG sensor, which captures electrical activity from different muscle regions.

S1-1: Performing 50 Hz notch filtering: power-line interference is removed.

S1-2: Performing band-pass filtering (30 Hz-500 Hz): Muscle characteristics are simulated to remove low-frequency and high-frequency noises, and retain valid EMG signals.

S2: Performing onset time detection in the offline mode: Obtaining onset time of motion intention T0_{offline} and an onset threshold mEMGₒₙₛₑₜ for subsequent online detection algorithms using a detEMG-based offline signal onset TO detection algorithm.

In S2, the onset time detection in the offline mode is performed using the detEMG-based onset time TO detection algorithm. detEMG of the signal is calculated using the algorithm to find onset time of sudden changes in the signal, thereby identifying the onset of motion intention. Specific steps are as follows:

S2-1: First, summing data from all channels, which is equivalent to compressing the high-density EMG signals into a single channel. On this basis, detEMG is calculated according to the following formula:

$${mEMG}_{i} = \frac{1}{N} {\sum }_{j = i}^{N + i - 1} \vert {x}_{j} \vert$$

$${detEMG}_{i} = {mEMG}_{i} - \frac{1}{{T}_{det}} {\int }_{{t}_{i} - {T}_{d e t}}^{{t}_{i}} {mEMG}_{i}$$

where N denotes a total number of data points; |xⱼ| denotes an absolute value of an i^{th} data point; mEMGᵢ denotes an EMG activation intensity value at the i^{th} data point; T_{det} denotes detection span time; and detEMGᵢ denotes a detection signal at the i^{th} data point.

S2-2: Calculating a time point at which the detEMGᵢ reaches 0.5 times its maximum value, and defining the same as the onset time of offline motion intention T0_{offline}.

S2-3: obtaining mEMGᵢ at the time point simultaneously, and recording as mEMGₒₙₛₑₜ, where the value will be subsequently used for online recognition.

S3: Performing signal frame formatting in the offline mode: Frame formatting is performed on the signals throughout a transient phase following the onset time TO. Starting from T0, signals of each channel are averaged over a fixed time interval ΔT between a t(i−1)^{th} time moment and a t(i) time moment, and the results are defined as an i^{th} frame signal (i=1, 2, 3 . . . f_sum). The specific calculation formula is as follows:

$${F}_{i} = \frac{1}{Δ t} {\int }_{{t}_{i} - Δ t}^{{t}_{i}} {mEmg}_{i}$$

where Fᵢ denotes the i^{th} frame signal (multi-channel data; it has an 8×8 matrix format when a sensor size is 8×8), and tᵢ=T₀+i×Δt denotes ending time of an i^{th} frame.

f_sum denotes a total number of frames throughout the transient phase, with a calculation formula as follows:

$$f\_sum = \frac{{T}_{transient}}{Δ t}$$

where Δt denotes a time interval, which may be set to 50-100 ms depending on a task; Tₜᵣₐₙₛᵢₑₙₜ denotes a duration of the entire transient phase, which may be set to an integer multiple of Δt in a range of 300-500 ms depending on the task.

S4: Extracting a feature in the offline mode: EMG activation intensity transition (directional activation intensity transition histogram) features for each signal frame are sequentially calculated. The specific calculation steps are as follows:

S4-1: Calculating the EMG activation intensity transition feature.

First, for each EMG sensor channel in the current frame, EMG activation intensity transition values in a horizontal direction STₓ and a vertical direction ST_{y} are calculated:

STₓ=mEMG(x+1,y)−mEMG(x−1,y)

ST_{y}=mEMG(x,y+1)−mEMG(x,y−1)

where mEMG(x,y) denotes an EMG activation intensity value at an EMG sensor channel (x, y) in the current frame;

an activation intensity transition magnitude ST and an activation intensity transition direction θ for each EMG sensor channel (x, y) are then calculated:

$$S T = \sqrt{S {T}_{x}^{2} + S {T}_{y}^{2}}$$

$$θ = {tan}^{- 1} \frac{S {T}_{y}}{S {T}_{x}}$$

S4-2: Constructing an EMG activation intensity transition histogram

The current frame is divided into a plurality of sub-frames, and each sub-frame contains 2×2 channels. Within each sub-frame, an activation intensity transition histogram is calculated based on the activation intensity transition direction of each channel. Specific steps are as follows:

S4-2-1: Dividing the activation intensity transition direction θ (0-180°) into a plurality of intervals.

S4-2-2: Assigning the activation intensity transition magnitude ST of each EMG sensor channel to a corresponding histogram bin according to its activation intensity transition direction θ. Specifically, when the activation intensity transition direction θ of the EMG sensor channel falls within a k^{th} interval, the activation intensity transition magnitude ST of the EMG sensor channel is added to a histogram of the k^{th} interval.

S4-2-3: converting the histogram into a feature vector, and obtaining a final EMG activation intensity transition feature vector.

S5: Training SVM classifiers: a plurality of classifiers corresponding to different analysis window lengths are trained in this stage.

S5-1: Classifying as training sets and test sets

All gesture sample data are classified as training sets and test sets according to a predetermined ratio, which may be selected within a range from 1:4 to 1:2.

S5-2: Inputting signals

Starting from an m^{th} frame (initial input frame), EMG activation intensity transition features from the m^{th} frame to an m+1^{th} frame are averaged across dimensions and input into the SVM classifiers.

S5-3: Obtaining prediction results and calculating confidence

For each gesture category, the SVM classifier outputs a prediction result and a corresponding confidence level, where the confidence level represents the reliability of the prediction result.

S5-4: Adjusting signal input

An analysis window length range of the SVM classifier is continuously extended, and S5-3 and S5-4 are repeated until all frames within the entire transient phase are covered.

S6: Acquiring and preprocessing signals in an online mode: EMG signals are acquired and preprocessed online using the high-density EMG sensor.

S6-1: Performing 50 Hz notch filtering: power-line interference is removed.

S6-2: Performing band-pass filtering (30 Hz-500 Hz): Muscle characteristics are simulated to remove low-frequency and high-frequency noises, and retain valid EMG signals.

S7: Performing signal onset time detection in the online mode: Onset time of motion intention is identified by adopting an mEMGₒₙₛₑₜ-based onset time T0ₒₙₗᵢₙₑ detection algorithm for an online signal.

S7-1: First, summing data from all channels, which is equivalent to compressing the high-density EMG signals into a single channel. On this basis, mEMGᵢ is calculated according to the following formula:

$${mEMG}_{i} = \frac{1}{N} {\sum }_{j = i}^{N + i - 1} \vert {x}_{j} \vert$$

where N denotes a total number of data points; |xⱼ| denotes an absolute value of an i^{th} data point; and mEMGᵢ denotes an EMG activation intensity value at the i^{th} data point.

S7-2: Comparing a current mEMGᵢ and mEMGₒₙₛₑₜ (a threshold at the onset time mEMGᵢ) until the current mEMGᵢ is greater than or equal to mEMGₒₙₛₑₜ, and marking the corresponding time point as an onset time T0ₒₙₗᵢₙₑ.

S8: Performing signal frame formatting in the online mode: Frame formatting is performed on the signals throughout a transient phase following the onset time T0ₒₙₗᵢₙₑ. Starting from T0ₒₙₗᵢₙₑ, signals of each channel are averaged over a fixed time interval ΔT between a t(i−1)^{th} time moment and a t(i) time moment, and the results are defined as an i^{th} frame signal (i=1, 2, 3 . . . f_sum), where f_sum denotes a total number of frames throughout the transient phase.

Refer to S3 for the specific calculation formula.

S9: Extracting a feature in the online mode: An EMG activation intensity transition (directional activation intensity transition histogram) feature is calculated for each signal frame.

Refer to S4 for the specific calculation method and formula.

S10: Inputting to an offline SVM classifier: Prediction type and confidence level of a current frame are calculated, and the analysis window length is adjusted dynamically.

S10-1: Calculating prediction type and confidence level of a current frame.

Starting from an m^{th} frame (initial input frame), EMG activation intensity transition features from the m^{th} frame to an m+1^{th} frame are averaged across dimensions and input into the corresponding offline SVM classifier. The current prediction Result and its confidence level Confidence are then calculated.

S10-2: Adjusting the analysis window length dynamically.

The confidence level Confidence obtained in the previous step is compared with a predefined confidence threshold Confidence_{accept}. When the confidence level reaches the predefined acceptable confidence level, the current prediction result is output, and further data acquisition is terminated. When the confidence level does not reach the predefined acceptable confidence level, a length of the input signal analysis window is increased, and S10-1 is repeated until the confidence level reaches the acceptable confidence level.

S10-3: Outputting the results

The recognition result is output, and further data acquisition is terminated. When the input signal length has already covered the entire transient phase, the recognition result is also output.

S11: Performing real-time feedback and adjustment: A confidence threshold is adjusted according to the real-time results.

S11-1: Presetting an initial confidence threshold Confidence_{accept_0}

S11-2: According to the real-time results, a confidence threshold Confidence_{accept_new} is adjusted to meet the needs of different individuals and tasks, thereby dynamically adjusting signal acquisition and analysis strategies and achieving a balance between recognition accuracy and response speed.

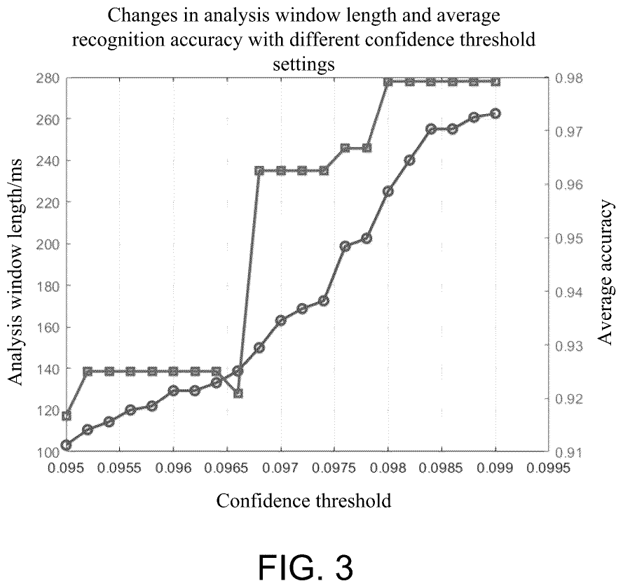

As shown in FIG. 3, changes in analysis window length and average recognition accuracy with different confidence threshold settings are illustrated in the embodiment of the present disclosure.

It should be noted that the above embodiments are provided for illustrative purposes only and are not intended to limit the scope of protection of the present disclosure. Equivalent modifications or substitutions made on the basis of the above technical solutions shall fall within the scope of protection defined by the appended claims.

What is claimed is:

1. A transient-phase EMG signal gesture recognition method based on an EMG activation intensity transition feature and an adaptive analysis window length, comprising the following steps:
S1: acquiring and preprocessing signals in an offline mode, wherein EMG signals are acquired and preprocessed offline using a high-density EMG sensor;
S2: performing onset time detection in the offline mode, wherein onset time of motion intention is identified by adopting a detEMG-based onset time TO detection algorithm;
S3: performing signal frame formatting in the offline mode, wherein frame formatting is performed on the signals throughout a transient phase following the onset time TO;
S4: extracting a feature in the offline mode, wherein EMG activation intensity transition features for each signal frame are sequentially calculated;
S5: training SVM classifiers, wherein a plurality of classifiers corresponding to different analysis window lengths are trained in this stage;
S6: acquiring and preprocessing signals in an online mode, wherein EMG signals are acquired and preprocessed online using the high-density EMG sensor;
S7: performing signal onset time detection in the online mode, wherein onset time of motion intention is identified by adopting an mEMGₒₙₛₑₜ-based onset time T0ₒₙₗᵢₙₑ detection algorithm for an online signal;
S8: performing signal frame formatting in the online mode, wherein frame formatting is performed on the signals throughout a transient phase following the onset time T0ₒₙₗᵢₙₑ;
S9: extracting a feature in the online mode, wherein EMG activation intensity transition features for each signal frame are sequentially calculated;
S10: inputting to an offline SVM classifier, wherein prediction type and confidence level of a current frame are calculated, and the analysis window length is adjusted dynamically; and
S11: performing real-time feedback and adjustment, wherein a confidence threshold is adjusted according to a real-time results.

2. The transient-phase EMG signal gesture recognition method based on the EMG activation intensity transition feature and the adaptive analysis window length according to claim 1, wherein in S1, the high-density EMG sensor is configured to provide EMG signals from 64 or more channels, so as to capture electrical activity from different muscle regions;
a method for preprocessing the signals in S1 comprises the following steps:
S1-1: performing 50 Hz notch filtering: power-line interference is removed;
S1-2: performing band-pass filtering (30 Hz-500 Hz): muscle characteristics are simulated to remove low-frequency and high-frequency noises, and retain valid EMG signals;
in S2, the onset time detection in the offline mode is performed using the detEMG-based onset time TO detection algorithm, wherein detEMG of the signal is calculated using the algorithm to find onset time of sudden changes in the signal, thereby identifying the onset of motion intention, with specific steps as follows:
S2-1: summing data from all channels, which is equivalent to compressing the high-density EMG signals into a single channel; and on this basis, detEMG is calculated according to the following formula:
$${mEMG}_{i} = \frac{1}{N} {\sum }_{j = i}^{N + i - 1} \vert {x}_{j} \vert$$
$${detEMG}_{i} = {mEMG}_{i} - \frac{1}{{T}_{det}} {\int }_{{t}_{i} - {T}_{d e t}}^{{t}_{i}} {mEMG}_{i}$$
wherein N denotes a total number of data points; |xⱼ| denotes an absolute value of an i^{th} data point; mEMGᵢ denotes an EMG activation intensity value at the i^{th} data point; T_{det} denotes detection span time; and detEMGᵢ denotes a detection signal at the i^{th} data point;
S2-2: calculating a time point at which the signal reaches 0.5 times a maximum value, and defining the same as the onset time TO; and
S2-3: obtaining mEMGᵢ at the time point simultaneously, and recording as mEMGₒₙₛₑₜ, wherein the value is subsequently used for online recognition.

3. The transient-phase EMG signal gesture recognition method based on the EMG activation intensity transition feature and the adaptive analysis window length according to claim 1, wherein a method for signal frame formatting throughout the transient phase in S3 is as follows: starting from T0, signals of each channel are averaged over a fixed time interval ΔT between a t(i−1)^{th} time moment and a t(i) time moment, and the results are defined as an i^{th} frame signal (i=1, 2, 3 . . . f_sum), with a calculation formula as follows:
$${F}_{i} = \frac{1}{Δ t} {\int }_{{t}_{i - Δ t}}^{{t}_{i}} {mEmg}_{i}$$
wherein Fᵢ denotes the i^{th} frame signal (multi-channel data; it has an 8×8 matrix format when a sensor size is 8×8), and tᵢ=T₀+i×Δt denotes ending time of an i^{th} frame;
f_sum denotes a total number of frames throughout the transient phase, with a calculation formula as follows:
$$f\_sum = \frac{{T}_{transient}}{Δ t}$$
wherein Δt denotes a time interval, which is set to 50-100 ms depending on a task; Tₜᵣₐₙₛᵢₑₙₜ denotes a duration of an entire transient phase, which is set to an integer multiple of Δt in a range of 300-500 ms depending on the task.

4. The transient-phase EMG signal gesture recognition method based on the EMG activation intensity transition feature and the adaptive analysis window length according to claim 1, wherein a method for extracting the feature in the offline mode in S4 is as follows: the EMG activation intensity transition features for the each signal frame are calculated; textural information from signals within local regions is extracted from the EMG activation intensity transition feature, and is suitable for describing spatial features of the EMG signals, and the EMG activation intensity transition feature has a strong capability to capture changes in muscle activity patterns, with calculation steps as follows:
S4-1: calculating the EMG activation intensity transition feature; wherein
for each EMG sensor channel in the current frame, EMG activation intensity transition values in a horizontal direction STₓ and a vertical direction ST_{y} are calculated:
STₓ=mEMG(x+1,y)−mEMG(x−1,y)
ST_{y}=mEMG(x,y+1)−mEMG(x,y−1)
wherein mEMG (x, y) denotes an EMG activation intensity value at an EMG sensor channel (x, y) in the current frame; and
an activation intensity transition magnitude ST and an activation intensity transition direction θ for each EMG sensor channel (x, y) are then calculated:
$$S T = \sqrt{S {T}_{x}^{2} + S {T}_{y}^{2}}$$
$$θ = {tan}^{- 1} \frac{S {T}_{y}}{S {T}_{x}}$$
S4-2: constructing an EMG activation intensity transition histogram, wherein the current frame is divided into a plurality of sub-frames, and each sub-frame comprises 2×2 channels; and
within each sub-frame, an activation intensity transition histogram is calculated based on the activation intensity transition direction of each channel, with specific steps as follows:
S4-2-1: dividing the activation intensity transition direction θ (0-180°) into a plurality of intervals;
S4-2-2: assigning the activation intensity transition magnitude ST of each EMG sensor channel to a corresponding histogram bin according to the activation intensity transition direction θ thereof; and when the activation intensity transition direction θ of the EMG sensor channel falls within a k^{th} interval, the activation intensity transition magnitude ST of the EMG sensor channel is added to a histogram of the k^{th} interval; and
S4-2-3: converting the histogram into a feature vector, and obtaining a final EMG activation intensity transition feature vector.

5. The transient-phase EMG signal gesture recognition method based on the EMG activation intensity transition feature and the adaptive analysis window length according to claim 1, wherein steps for training the SVM classifiers in S5 are as follows:
S5-1: classifying as training sets and test sets, wherein all gesture sample data are classified as the training sets and the test sets according to a predetermined ratio, and the predetermined ratio is selected within a range from 1:4 to 1:2;
S5-2: inputting the signals, wherein starting from an m^{th} frame (initial input frame), the EMG activation intensity transition features from the m^{th} frame to an m+1^{th} frame are averaged across dimensions and input into the SVM classifiers;
S5-3: obtaining prediction results and calculating confidence, wherein for each gesture category, the SVM classifier outputs a prediction result and a corresponding confidence level, and the confidence level represents the reliability of the prediction result; and
S5-4: adjusting signal input, wherein an analysis window length range of the SVM classifier is continuously extended, and S5-3 and S5-4 are repeated until all frames within an entire transient phase are covered.

6. The transient-phase EMG signal gesture recognition method based on the EMG activation intensity transition feature and the adaptive analysis window length according to claim 1, wherein a method for preprocessing the signals in S6 comprises the following steps:
S6-1: performing 50 Hz notch filtering: power-line interference is removed;
S6-2: performing band-pass filtering (30 Hz-500 Hz): muscle characteristics are simulated to remove low-frequency and high-frequency noises, and retain valid EMG signals;
steps for adopting the mEMGₒₙₛₑₜ-based onset time T0ₒₙₗᵢₙₑ detection algorithm in S7 are as follows:
S7-1: summing data from all channels, which is equivalent to compressing a high-density EMG signals into a single channel; and on this basis, mEMGᵢ is calculated according to the following formula:
$${mEMG}_{i} = \frac{1}{N} {\sum }_{j = i}^{N + i - 1} \vert {x}_{j} \vert$$
wherein N denotes a total number of data points; |xⱼ| denotes an absolute value of an i^{th} data point; and mEMGᵢ denotes an EMG activation intensity value at the i^{th} data point; and
S7-2: comparing a current mEMGᵢ and mEMGₒₙₛₑₜ (a threshold at the onset time mEMGᵢ) until the current mEMGᵢ is greater than or equal to the mEMGₒₙₛₑₜ, and marking the corresponding time point as an onset time T0ₒₙₗᵢₙₑ.

7. The transient-phase EMG signal gesture recognition method based on the EMG activation intensity transition feature and the adaptive analysis window length according to claim 1, wherein a method for the signal frame formatting in S8 is as follows: starting from T0ₒₙₗᵢₙₑ, signals of each channel are averaged over a fixed time interval ΔT between a t(i−1)^{th} time moment and a t(i) time moment, and the results are defined as an i^{th} frame signal (i=1, 2, 3 . . . f_sum), wherein f_sum denotes a total number of frames throughout the transient phase; and a calculation formula as follows:
$$f\_sum = \frac{{T}_{transient}}{Δ t}$$
wherein Δt denotes a time interval, and Tₜᵣₐₙₛᵢₑₙₜ denotes a duration of the entire transient phase, which remains consistent with the offline mode; and
a method for extracting the feature in the online mode in S9 is to calculate the EMG activation intensity transition features for the each signal frame.

8. The transient-phase EMG signal gesture recognition method based on the EMG activation intensity transition feature and the adaptive analysis window length according to claim 1, wherein steps for inputting to the offline SVM classifier in S10 are as follows:
S10-1: calculating prediction type and confidence level of a current frame; wherein
starting from an m^{th} frame (initial input frame), the EMG activation intensity transition features from the m^{th} frame to an m+1^{th} frame are averaged across dimensions and input into the corresponding SVM classifier, and a current prediction Result and a confidence level Confidence thereof are then calculated;
S10-2: adjusting the adaptive analysis window length dynamically; wherein
the confidence level Confidence obtained in the previous step is compared with a predefined confidence threshold Confidence_{accept}; when the confidence level reaches a predefined acceptable confidence level, a current prediction result is output, and further data acquisition is terminated; and when the confidence level does not reach the predefined acceptable confidence level, a length of an input signal analysis window is increased, and S10-1 is repeated until the confidence level reaches the predefined acceptable confidence level; and
S10-3: outputting results, wherein recognition results are output, and further the data acquisition is terminated; and when an input signal length has already covered an entire transient phase, the recognition results are also output.

9. The transient-phase EMG signal gesture recognition method based on the EMG activation intensity transition feature and the adaptive analysis window length according to claim 1, wherein a specific method for performing the real-time feedback and adjustment in S11 is as follows:
S11-1: presetting an initial confidence threshold Confidence_{accept}_o; and
S11-2: adjusting a confidence threshold Confidence_{accept}_new according to the real-time results to meet needs of different individuals and tasks, thereby dynamically adjusting signal acquisition and analysis strategies and achieving a balance between recognition accuracy and response speed.

10. An electronic device, comprising a memory, a processor and a computer program stored on the memory and runnable on the processor, wherein the processor is configured to implement the transient-phase EMG signal gesture recognition method based on the EMG activation intensity transition feature and the adaptive analysis window length according to claim 1.
